(19) United States
(12) Patent Application Publication
(10) Pub. No.: US 2023/0233880 A1
(43) Pub. Date: Jul. 27, 2023

(54) ACOUSTIC MIRROR

(71) Applicant: HEALIUM MEDICAL LTD., Yokneam (IL)

(72) Inventors: Ran Sela, Ramat Hasharon (IL); Yuri Megel, Haifa (IL)

(21) Appl. No.: 18/002,360

(22) PCT Filed: Jun. 20, 2021
(86) PCT No.: PCT/IB2021/055422

§ 371 Date: Dec. 19, 2022

Related U.S. Application Data
(60) Provisional application No. 63/043,832, filed on Jun. 25, 2020.

(51) Int. Cl. A61N 7/02; A61B 17/32

(52) U.S. Cl. CPC A61N 7/022; A61B 17/320068; A61N 2007/0069

(57) ABSTRACT

In accordance with some embodiments of the invention there is provided an acoustic mirror (200, 600, 700, 800), including an ultrasound beam deflecting surface (306) and a base couplable to an ultrasound catheter (102) comprising an ultrasound beam emitting surface (104). When the acoustic mirror is mounted on the ultrasound catheter the deflecting surface is angled in relation to ultrasound beam emitting surface and configured to (i) at least partially interfere with a path of propagation of an ultrasound beam mitted from the emitting surface and (ii) deflect the ultrasound beam impinging on the deflecting surface. Other embodiments are also described.

### CROSS-REFERENCES TO RELATED APPLICATIONS

The present application claims priority from U.S. Provisional Patent Application No. 63/043,832 to Sela et al., filed Jun. 25, 2020, entitled “Acoustic Mirror”, which is incorporated herein by reference.

### FIELD OF THE INVENTION

Some embodiments of the present invention generally relate to devices and methods for treatment of tissue by application of energy thereto, and more specifically to ablation of cardiac tissue by application of ultrasound energy for treating cardiac arrhythmias, such as atrial fibrillation.

### BACKGROUND

Atrial fibrillation is a common cardiac arrhythmia involving the atria of the heart. During atrial fibrillation, the atria beat irregularly and out of coordination with the ventricles of the heart, thereby disrupting efficient beating of the heart. Atrial fibrillation symptoms often include heart palpitations, shortness of breath and weakness. A major concern with atrial fibrillation is the potential to develop blood clots within the atria of the heart. These blood clots forming in the heart may circulate to other organs and lead to serious medical conditions such as strokes.

Atrial fibrillation is generally caused by abnormal electrical activity in the heart. During atrial fibrillation, electrical discharges may be generated by parts of the atria which do not normally generate electrical discharges, such as pulmonary vein ostia in the atrium.

Ablation procedures, e.g., catheter ablation, are generally used to terminate a faulty electrical pathway from sections of the heart, especially in those who are prone to developing cardiac arrhythmias and to restore the heart to its normal rhythm. For example, pulmonary vein isolation by ablation is a common medical procedure for treatment of atrial fibrillation.

The foregoing examples of the related art and limitations related therewith are intended to be illustrative and not exclusive. Other limitations of the related art will become apparent to those of skill in the art upon a reading of the specification and a study of the figures.

### SUMMARY

There is provided in accordance with some embodiments of the present invention, apparatus for use with a lumen, e.g., a pulmonary vein, that extends from a heart chamber, e.g., the left atrium of the heart. Typically, the apparatus applies ultrasound energy to ablate tissue of the ostium of the pulmonary vein to electrically isolate the pulmonary vein to treat cardiac arrhythmia. For some embodiments, the apparatus comprises a transluminal ablation catheter (also referred to herein as an ultrasound catheter) comprising at least one ultrasound transducer that is typically coupled to a distal portion of the catheter and configured to be inserted into the chamber of the subject's heart. The ultrasound transducer comprises at least one ultrasound emitting surface configured to emit an ultrasound beam to ablate cardiac tissue of the subject. For some embodiments, the apparatus additionally comprises an ultrasound beam guiding, e.g., deflecting, element coupled to the catheter and having an ultrasound beam guiding, e.g., deflecting, surface. The ultrasound beam deflecting surface is positioned in relation to the ultrasound beam emitting surface so as to at least partially interfere with a path of propagation of the emitted ultrasound beam such that the ultrasound beam is directed from the deflecting element towards an ostium of the lumen (e.g., of the pulmonary vein) to ablate tissue of the ostium of the lumen.

In the present application, the ultrasound beam deflecting element is sometimes referred to as an acoustic mirror, and the terms “ultrasound beam guiding element” and “ultrasound beam deflecting element” is used interchangeably with, the term “acoustic mirror”. This is because the ultrasound beam guiding element acts to redirect acoustic waves in a similar manner to a mirror that redirects light waves. It is noted that the term “acoustic mirror” should not be interpreted as implying a particular shape that such a term may be commonly understood to imply. Rather, any limitations regarding the shape of the acoustic mirror are solely as described herein. For some embodiments, the acoustic mirror includes a base through which the acoustic mirror is coupled to the ultrasound catheter. The acoustic mirror is typically coupled to the ultrasound catheter such that the ultrasound beam deflecting surface is angled in relation to the ultrasound beam emitting surface. The ultrasound beam deflecting surface is configured to (i) at least partially interfere with a path of propagation of an ultrasound beam emitted from the emitting surface, and (ii) deflect the ultrasound beam impinging on the deflecting surface. In some embodiments, the deflecting surface is pivotable about the base. In some embodiments, the base is resilient.

According to some embodiments of the present invention, a geometry of the ultrasound beam deflecting surface at least partially overlaps with a geometry of the ultrasound beam emitting surface. In some embodiments, when the acoustic mirror is mounted on the ultrasound catheter, the deflecting surface is pivotable about the base from a closed state in which the deflecting surface is parallel to a wall of the ultrasound catheter, to an open state in which the deflecting surface is angled (i.e., not parallel) in relation to the emitting surface. In some embodiments, an angle between the deflecting surface and the emitting surface is between 0 (zero) and 75 degrees. In some embodiments, the deflecting surface is concave along its short axis. In some embodiments, the acoustic mirror also includes an air chamber.

According to some embodiments of the present invention, the acoustic mirror also includes a cooling mechanism. In some embodiments, the cooling mechanism includes at least one or a combination of a heat sink, a cooling fluid channel, a coating of a material having a high heat conductivity coefficient and a cooling capsule. In some embodiments, the acoustic mirror is coated with an anticoagulant. In some embodiments, the acoustic mirror is encapsulated with air to increase energy reflection and prevent energy absorbent within the mirror material. In some embodiments, the deflecting surface has a semi-cone or parabola-like geometry. In some embodiments, side-edges of the acoustic mirror diverge laterally outwards at a divergence angle corresponding to a divergence angle of the ultrasound beam from borders of the US emitting surface.

In accordance with additional embodiments of the present invention, the apparatus comprises an ultrasound beam guiding element, e.g., an acoustic mirror add-on, for use with an ultrasound catheter. For such embodiments, the acoustic mirror does not form an integral part of the ultrasound catheter, but rather is removably couplable to the ultrasound catheter. Typically, the acoustic mirror add-on includes an ultrasound beam deflecting surface, an ultrasound catheter coupling element, e.g., a mounting ring, couplable to the ultrasound catheter, and a base coupling the deflecting surface and the mounting ring. Typically, when the acoustic mirror add-on is coupled to the ultrasound catheter, the deflecting surface is angled in relation to the ultrasound beam emitting surface of the ultrasound transducer so that when the acoustic mirror is positioned such that the deflecting surface faces the source of ultrasound energy and at least partially interferes with a path of propagation of the ultrasound beam emitted by the emitting surface, the ultrasound beam is deflected from the path of propagation.

In some embodiments, the base coupling the deflecting surface and the mounting ring is resilient. In some embodiments, the mounting ring is mountable on a standard ultrasound catheter. For example, the mounting ring is slidingly mountable on a standard ultrasound catheter. In some embodiments, the mounting ring is rotatable about a longitudinal axis of the ultrasound catheter. In some embodiments, the mounting ring is mountable on an ultrasound catheter having a rotating ultrasound transceiver and is configured to rotate about the catheter in correspondence with rotation of the US transceiver. In some embodiments, the mounting ring is slidable along the ultrasound catheter. In some embodiments, an angle between the deflecting surface and the emitting surface of the ultrasound transducer of the ultrasound catheter is between 0 (zero) and 75 degrees.

In accordance with some embodiments of the present invention an ultrasound catheter acoustic mirror system, is provided. Typically, the system includes an ultrasound catheter including an ultrasound transducer having at least one ultrasound emitting surface, and an acoustic mirror having an ultrasound beam deflecting surface and being coupled to the ultrasound catheter via a base. The acoustic mirror is mounted on the ultrasound catheter such that the deflecting surface is angled in relation to the ultrasound beam emitting surface and configured to (i) at least partially interfere with a path of propagation of an ultrasound beam emitted from the emitting surface, and (ii) deflect the ultrasound beam impinging on the deflecting surface. In accordance with some embodiments, the deflecting surface is pivotable about the base. In some embodiments, the base is resilient. In some embodiments, a geometry of the ultrasound beam deflecting surface at least partially overlaps a geometry of the ultrasound beam emitting surface. In some embodiments, the deflecting surface is pivotable about the base from a closed state in which the deflecting surface is parallel to a wall of the ultrasound catheter, to an open state in which the deflecting surface is angled in relation to the emitting surface.

In accordance with some embodiments, the system additionally includes an acoustic mirror control mechanism. In some embodiments, the control mechanism is configured to move the acoustic mirror from a closed state, in which the deflecting surface is parallel to a wall of the ultrasound catheter, to an open state in which the deflecting surface is angled in relation to the emitting surface. In some embodiments, the control mechanism is configured to control an angle between the deflecting surface of the acoustic mirror and the emitting surface of the ultrasound transducer of the ultrasound catheter. In some embodiments, angle is between 0 (zero) and 75 degrees. In some embodiments, the control mechanism is a slidable retainer. In some embodiments, the slidable retainer includes a sleeve. In some embodiments, the control mechanism is a slidable wire coupled to the base of the acoustic mirror via a slidable arm.

There is therefore provided, in accordance with some applications of the present invention, an acoustic mirror for use with an ultrasound catheter that includes an ultrasound beam emitting surface, including:
an ultrasound beam deflecting surface; and
a base couplable to the ultrasound catheter,
when the acoustic mirror is mounted on the ultrasound catheter, the deflecting surface being angled in relation to the ultrasound beam emitting surface and configured to:
   (i) at least partially interfere with a path of propagation of an ultrasound beam emitted from the emitting surface; and
   (ii) deflect the ultrasound beam impinging on the deflecting surface.

For some applications, the ultrasound beam deflecting surface is pivotable about the base.

For some applications, the base is resilient.

For some applications, a geometry of the ultrasound beam deflecting surface at least partially overlaps a geometry of the ultrasound beam emitting surface.

For some applications, when mounted on the ultrasound catheter, the deflecting surface is pivotable about the base from a closed state in which the deflecting surface is parallel to a wall of the ultrasound catheter, to an open state in which the deflecting surface is angled in relation to the emitting surface.

For some applications, the deflecting surface is pivotable about the base such that an angle between the deflecting surface and the emitting surface is changeable at least from 0 to 75 degrees.

For some applications, the deflecting surface defines a short axis and is concave along its short axis.

For some applications, the acoustic mirror further includes an air chamber.

For some applications, the acoustic further includes a cooling mechanism.

For some applications, the cooling mechanism includes at least one or a combination of a heat sink, a cooling fluid channel, a coating of a material having a high heat conductivity coefficient and a cooling capsule.

For some applications, the acoustic mirror is coated with an anticoagulant.

For some applications, the acoustic mirror is encapsulated in air to increase energy reflection and inhibit energy absorbent within the mirror material.

For some applications, the deflecting surface has a semi-cone or parabola-like geometry.

For some applications, side-edges of the acoustic mirror diverge laterally outwards at a divergence angle corresponding to a divergence angle of an ultrasound beam from borders of the ultrasound emitting surface.

There is further provided, in accordance with some applications of the present invention, an acoustic mirror add-on for use with ultrasound catheter that includes a source of ultrasound energy and an ultrasound beam emitting surface, including:
an ultrasound beam deflecting surface;
an ultrasound catheter mounting ring couplable to the ultrasound catheter; and
a base coupling the deflecting surface and the mounting ring; and
the deflecting surface being angled in relation to the emitting surface so that when the acoustic mirror is positioned such that the deflecting surface faces the source of ultrasound energy and at least partially interferes with a path of propagation of an ultrasound beam emitted by the emitting surface, the ultrasound beam is deflected from the path of propagation.

For some applications, the base is resilient.

For some applications, the mounting ring is mountable on a standard ultrasound catheter.

For some applications, the mounting ring is slidingly mountable on a standard ultrasound catheter.

For some applications, the mounting ring is rotatable about a longitudinal axis of the ultrasound catheter.

For some applications, the mounting ring is mountable on an ultrasound catheter having a rotating ultrasound transceiver and is configured to rotate about the ultrasound catheter in correspondence with rotation of the ultrasound transceiver.

For some applications, the mounting ring is slidable along the ultrasound catheter.

For some applications, an angle between the deflecting surface and the emitting surface is between 0 (zero) and 75 degrees.

There is further provided, in accordance with some applications of the present invention, an ultrasound catheter acoustic mirror system, including:
an ultrasound catheter including an ultrasound emitting surface; and
an acoustic mirror including an ultrasound beam deflecting surface, coupled to the ultrasound catheter via a base; and
wherein the acoustic mirror is coupled to the ultrasound catheter such that the deflecting surface is angled in relation to the ultrasound beam emitting surface and configured to:
   (i) at least partially interfere with a path of propagation of an ultrasound beam emitted from the emitting surface; and
   (ii) deflect the ultrasound beam impinging on the deflecting surface.

For some applications, the deflecting surface is pivotable about the base.

For some applications, the base is resilient.

For some applications, a geometry of the ultrasound beam deflecting surface at least partially overlaps a geometry of the ultrasound beam emitting surface.

For some applications, the deflecting surface is pivotable about the base from a closed state in which the deflecting surface is parallel to a wall of the ultrasound catheter, to an open state in which the deflecting surface is angled in relation to the emitting surface.

For some applications, the system further includes an acoustic mirror control mechanism.

For some applications, the control mechanism is configured to move the acoustic mirror from a closed state, in which the deflecting surface is parallel to a wall of the ultrasound catheter, to an open state in which the deflecting surface is angled in relation to the emitting surface.

For some applications, the control mechanism is configured to control an angle between the deflecting surface and the emitting surface.

For some applications, the angle is between 0 and 75 degrees.

For some applications, the control mechanism includes a slidable retainer.

For some applications, the slidable retainer includes a sleeve.

For some applications, the control mechanism includes a slidable wire coupled to the base of the acoustic mirror via a slidable arm.

For some applications, the deflecting surface defines a short axis and is concave along its short axis.

For some applications, the acoustic mirror further includes an air chamber.

For some applications, the acoustic mirror further includes a cooling mechanism.

For some applications, the cooling mechanism includes at least one or a combination of a heat sink, a cooling fluid channel, a coating of a material having a high heat conductivity coefficient and a cooling capsule.

For some applications, the deflecting surface has a semi-cone or parabola-like geometry.

For some applications, side-edges of the acoustic mirror diverge laterally outwards at a divergence angle corresponding to a divergence angle of an ultrasound beam from borders of the ultrasound emitting surface.

There is further provided, in accordance with some applications of the present invention, apparatus for use with a lumen of a subject that extends from a chamber of a heart of the subject, the apparatus including:
a transluminal ablation catheter including at least one ultrasound transducer (i) having at least one ultrasound beam emitting surface, and (ii) configured to be inserted into the chamber of the subject's heart and to emit an ultrasound beam from the ultrasound beam emitting surface; and
an ultrasound beam guiding element coupled to the transluminal ablation catheter and including an ultrasound beam guiding surface configured to be positioned in relation to the ultrasound beam emitting surface so as to at least partially interfere with a path of propagation of the emitted ultrasound beam such that the ultrasound beam is directed from the guiding element toward an ostium of the lumen to ablate tissue of the ostium of the lumen.

For some applications, the ultrasound beam emitting surface is configured such that ultrasound beam is directed from the ultrasound beam guiding surface from a distance that is within 3-6 mm from the tissue of the ostium of the lumen.

For some applications, the at least one ultrasound transducer is configured to be inserted into a left atrium in a vicinity of a pulmonary vein ostium and is configured to ablate tissue of an ostium of the pulmonary vein, to thereby electrically isolate the pulmonary vein.

The present invention will be more fully understood from the following detailed description of embodiments thereof, taken together with the drawings, in which:

### BRIEF DESCRIPTION OF THE FIGURES

FIG. 1 is a perspective view schematic illustration of an ultrasound catheter of the type that is adapted to incorporate an acoustic mirror, in accordance with some embodiments of the invention;

FIGS. 2A and 2B are cross section view schematic illustrations of an ultrasound catheter of the type that is adapted to incorporate an acoustic mirror, according to some embodiments of the invention;

FIGS. 3A, 3B and 3C are a perspective view and cross-section views schematic illustrations of an ultrasound catheter with an acoustic mirror in accordance with some embodiments of the invention;

FIGS. 4A, 4B, 4C, 4D, 4E, 4F, 4G, 4H and 4I are cross section schematic illustrations of examples of acoustic mirror control mechanisms, in accordance with some embodiments of the invention;

FIGS. 5A, 5B, 5C, 5D, 5E and 5F are perspective view and cross section view schematic illustrations of acoustic mirror add-ons for use with an ultrasound catheter, in accordance with some embodiments of the invention;

FIGS. 6A and 6B are cross section view and perspective view schematic illustrations of behavior of an emitted ultrasound beam and an acoustic mirror, in accordance with some embodiments of the invention;

FIGS. 6C and 6D are perspective view schematic illustrations of an acoustic mirror add-on, in accordance with some embodiments of the invention;

FIGS. 7A and 7B are a perspective view and cross-section view of an acoustic mirror add-on for use with an ultrasound catheter, in accordance with some embodiments of the invention;

FIG. 8 is a perspective view schematic illustration of an acoustic mirror add-on with a concave acoustic mirror, in accordance with some embodiments of the invention;

FIG. 9 is a cross-section view schematic illustration of a concave deflective surface induced jet effect, in accordance with some embodiments of the invention; and

FIG. 10 is a perspective view schematic illustration of an acoustic mirror add-on cooling mechanism, in accordance with some embodiments of the invention.

### DETAILED DESCRIPTION

According to some aspects of the invention there is provided a transluminal ultrasound catheter comprising an ultrasound transducer sized and fitted to be positioned along the catheter and/or within a delivery catheter. In some embodiments, the ultrasound transducer is an ablative ultrasound transducer. In some embodiments, the ultrasound transducer is mounted on the catheter such that the ultrasound transmitting surface faces generally laterally such that emitted ultrasound beam is directed perpendicular to, or at an angle, to the vessel walls. In some embodiments, the ultrasound catheter comprises an acoustic mirror positioned such as to deflect the ultrasound beam from its original path of propagation. The acoustic mirror is configured to deflect the ultrasound beam to a desired new path of propagation at any angle relative to a longitudinal axis of the ultrasound catheter.

As noted hereinabove, in the present application, the term “acoustic mirror” should be interpreted as referring to an ultrasound beam guiding/deflecting element, which acts to redirect acoustic waves in a similar manner to a mirror that redirects light waves. It is noted that the term “acoustic mirror” should not be interpreted as implying a particular shape that such a term may be commonly understood to imply. Rather, any limitations regarding the shape of the acoustic mirror are solely as described herein.

According to some aspects of the invention, an acoustic mirror control mechanism is provided, which is configured to position the acoustic mirror in relation to the longitudinal axis of the ultrasound catheter. Typically, the control mechanism is configured to translate the acoustic mirror axially along the longitudinal axis of the ultrasound catheter. In some embodiments, the control mechanism may position the acoustic mirror e.g., proximally, distally, or at any point in between along a length of the ultrasound catheter.

According to some aspects of the invention, the acoustic mirror control mechanism is configured to move the acoustic mirror between a closed position and an open position. Additionally, or alternatively, the acoustic mirror control mechanism is configured to control an angle of the acoustic mirror in relation to the longitudinal axis of the ultrasound catheter, and thus control the angle of deflection of the ultrasound beam by the acoustic mirror.

According to some aspects of the invention, the acoustic mirror control mechanism comprises a slidable retainer or a slidable sleeve slidable proximally and/or distally to control an angle of the acoustic mirror in relation to the longitudinal axis of the ultrasound catheter, and thus control the angle of deflection of the ultrasound beam by the acoustic mirror.

According to some aspects of the invention, the acoustic mirror is rotatably fixed in relation to the ultrasound catheter. In some embodiments, the acoustic mirror is fixed, connected to, and/or attached to the ultrasound transducer. In some embodiments, the acoustic mirror is rotatable about the longitudinal axis of the ultrasound catheter. In some embodiments, the acoustic mirror is mounted on a sleeve rotatable about the longitudinal axis of the ultrasound catheter. In some embodiments, the acoustic mirror control mechanism is configured to control the rotation of the rotatable sleeve. In some embodiments, the acoustic mirror and the transducer are each adapted to rotate. In some embodiments, the acoustic mirror and the transducer are adapted to rotate in tandem.

According to some aspects of the invention, the acoustic mirror control mechanism is configured to control the rotation of the acoustic mirror about itself. In some embodiments, the acoustic mirror is configured to be positioned at any location along a border of an ultrasound transducer emitting surface. In some embodiments, the acoustic mirror is mountable proximally to the ultrasound transducer emitting surface, thereby deflecting emitted ultrasound beams distally. In some embodiments, the acoustic mirror is mountable distally to the ultrasound transducer emitting surface, thereby deflecting emitted ultrasound beams proximally. In some embodiments, the acoustic mirror is mountable alongside a lateral border of the ultrasound transducer emitting surface, thereby deflecting emitted ultrasound beams laterally.

According to some aspects of the invention there is provided an acoustic mirror add-on for an ultrasound catheter. In some embodiments, the acoustic mirror add-on comprises an acoustic mirror coupled to a mounting ring via a base. In some embodiments, the acoustic mirror add-on comprises an acoustic mirror coupled to a mounting ring integrally formed of any suitable material, e.g., nitinol. In some embodiments, the base connecting the acoustic mirror and the mounting ring is resilient. In some embodiments, the base is made of a shape-memory alloy. In some embodiments, the acoustic mirror add-on is couplable to any conventional ultrasound catheter e.g., by sliding the mounting ring over the catheter to a position in which the geometry of beam deflecting surface corresponds to the geometry of ultrasound transducer emitting surface so that to capture most, and possibly all of, the acoustic energy emitted from the ultrasound transducer. In some embodiments, the add-on is rotatable.

According to an aspect of some embodiments of the invention there is provided an ablative US transducer catheter comprising an acoustic mirror positioned such as to deflect the US beam from its original path of propagation. In some embodiments, the ultrasound transducer is an ablative ultrasound transducer.

In accordance with some aspects of the inventions, the acoustic mirrors described herein comprise a heat dissipating mechanism. In some embodiments, the heat dissipating mechanism comprises a heat sink. In some embodiments, the heat dissipating mechanism comprises heat removal cooling liquid channels.

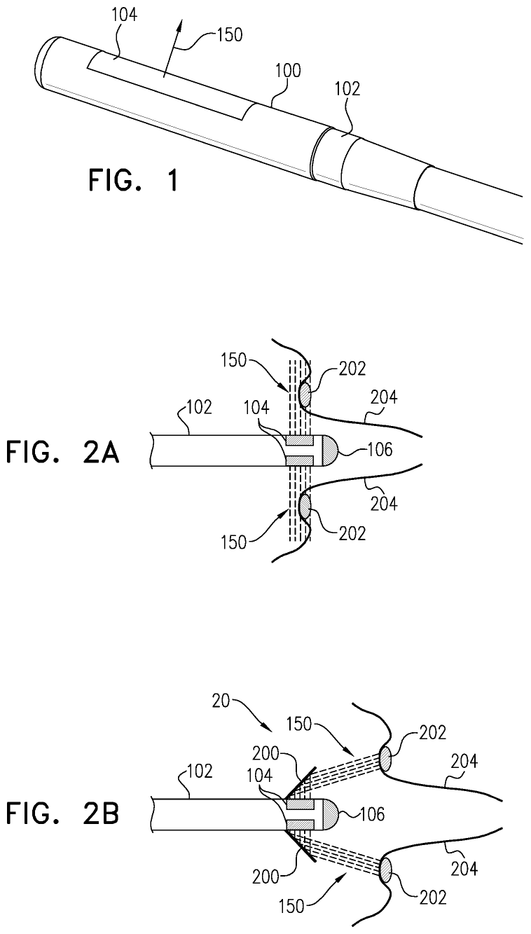

Reference is now made to FIGS. 1, 2A and 2B, which are a perspective view and cross section view schematic illustrations of an ultrasound catheter 102 the type of which is adapted to incorporate an acoustic mirror, ultrasound catheter 102 comprising a laterally emitting ultrasound transducer 100 (FIGS. 1 and 2A), and an ultrasound catheter acoustic mirror system 20, according to some embodiments of the invention (FIG. 2B). As shown, ultrasound transducer 100 is attached to a tip of ultrasound catheter 102, e.g., as described in PCT Patent Application Publication No. WO 2020/039442, which is incorporated herein by reference. For some applications, the catheter is functionally coupled to one or more sources of cooling fluid, power (e.g., electric power), vacuum and unidirectional and/or bidirectional data communication conduits. The term “Cooling Fluid” as used herein relates to a fluid having a temperature that will maintain a temperature of a blood-contact surface of ultrasound transducer 100 no higher than that of the surrounding blood temperature.

Though the acoustic mirror as disclosed herein is described mainly in the context of ultrasound ablation treatment, e.g., employing an ablating ultrasound energy beam greater than 15 W/cm∧2, the disclosed acoustic mirror is operable with any ultrasound beam emitting surface. Hence, the term “ultrasound transceiver”, as used herein refers to any one of an ablative energy ultrasound transmitter, an imaging ultrasound beam transmitter, an ultrasound beam receiver or any combination thereof.

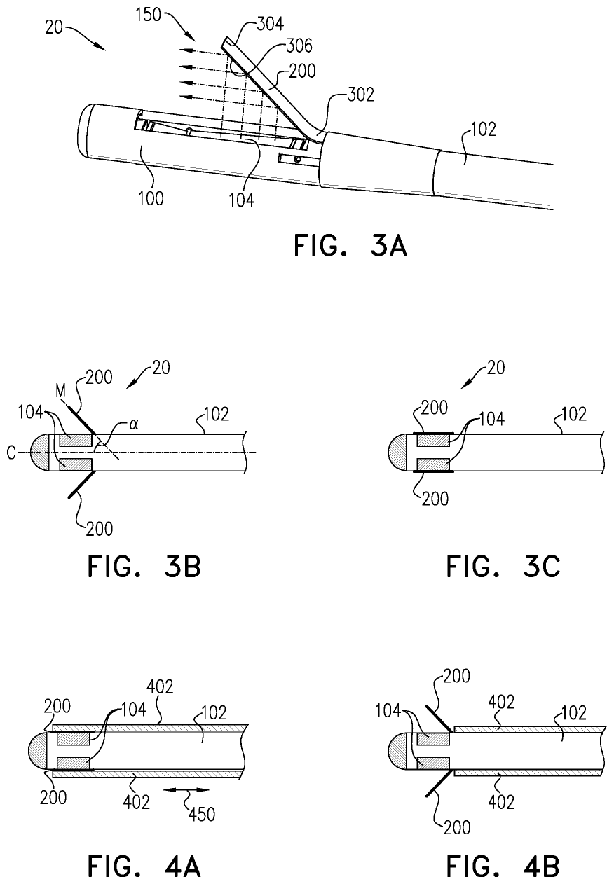

As used herein the term “proximal” means closer to the user of the ultrasound catheter and farther from the tip of the ultrasound catheter (which is advanced furthest into the subject's body) and the term “distal” means closer to the tip of the ultrasound catheter (which is advanced furthest into the subject's body) and farther from the user of the ultrasound catheter. FIG. 1, shows an emitting surface 104 of ultrasound transducer 100 that faces laterally such that ultrasound beam 150, is emitted generally perpendicularly to emitting surface 104 and generally perpendicularly to a longitudinal axis of ultrasound transducer 100 and catheter 102 (indicated by longitudinal axis C shown in FIG. 3B). In general, the angle of ultrasound beam 150 in relation with the longitudinal axis of ultrasound transducer 100 and catheter 102 depends on the mounting position of ultrasound transducer 100 on catheter 102. For example, in some embodiments, ultrasound transducer 100 is mounted on catheter 102 at an angle such that an ultrasound beam emitted from emitting surface 104 of ultrasound transducer 100 is angled in relation to the longitudinal axis of the ultrasound transducer 100 and catheter 102. However, the angles at which US transducer 100 can be positioned are somewhat limited.

FIG. 2A illustrates an exemplary use of ultrasound catheter 102, in contactless ablation of ostia of the pulmonary veins in the left atrium of the heart. In some cases, such as, for example atrial fibrillation treatment, the pulmonary vein is ablated to stop the electrical trigger. In this procedure, the ultrasound catheter is generally centered in the ostium to allow safe ablation of the ostium margins. As depicted in FIG. 2A, a side-emitting ultrasound transducer 100 ultrasound mounted on catheter 102 is positioned within the ostium so that the ultrasound beams 150 impinge effectively on the ostia margins 202 of the pulmonary vein ostium.

However, at times, such a positioning requires bringing the ultrasound transducer in close proximity to the tissue to be treated risking damage to surrounding tissue. Moreover, such positioning sometimes limits the angle of impingement of the ultrasound beam on the tissue, which may not be an optimal angle for effective treatment. Commonly, it may be possible to overcome these potential drawbacks by using a positioner, e.g., a cage-shaped positioner to stabilize the ultrasound catheter within the ostium, to maintain a correct positioning of the ultrasound catheter in relation to the tissue to be treated as well as maintain a safe distance between the ultrasound emitting surface and the tissue e.g., vessel/lumen wall.

Reference is now made to FIG. 2B, which is a schematic illustration of ultrasound catheter acoustic mirror system 20, in accordance with some embodiments of the present invention. In some embodiments, system 20 comprises ultrasound catheter 102, which is typically a transluminal ablation catheter comprising at least one ultrasound transducer 100 coupled to a distal portion of catheter 102 and having one or more ultrasound beam emitting surfaces 104. As shown, ultrasound transducer 100 is configured to be brought in vicinity of a target tissue, e.g., ostia of pulmonary vein tissue 204. Ultrasound emitting surface 104 is configured to emit ultrasound beam 150 to ablate pulmonary vein ostia. System 20 additionally comprises an ultrasound beam guiding element, e.g., an acoustic mirror 200.

Acoustic mirror 200 is typically positioned at an angle in relation to the ultrasound emitting surface 104 of transducer 100 such as to interfere with a path of propagation of ultrasound beam 150 emitted from US emitting surface 104 and deflects ultrasound beam 150 emitted from US emitting surface 104 towards tissue to be ablated.

As shown in FIG. 2A, ultrasound beam 150 emitted laterally from ultrasound emitting surface 104 of transducer 100 propagates generally forward, past a tip 106 of ultrasound catheter 102 and angled outwards, away from the longitudinal axis of ultrasound catheter 102. A potential advantage of acoustic mirror 200, shown in FIG. 2B, is in that ultrasound beam 150 is directed at any desirable angle in relation to ultrasound transducer 100 which negates the need to position ultrasound transducer 100 in close proximity to the tissue to be treated, and therefore the need for a positioner, as well as provides for an optimal angle of impingement of the emitted beam on the tissue. In some embodiments, acoustic mirror 200 is up to, but not limited to 15 mm in length and 3 mm in width. In some embodiments, the ultrasound beam emitting surface is configured such that ultrasound beam is directed from the ultrasound beam guiding surface from a distance that is within, 1-10 mm, e.g., 2-8 mm, e.g., 3-6 mm, from the tissue of the ostium of the lumen.

As is explained in greater detail elsewhere herein, in some embodiments, ultrasound beam 150, which is deflected by acoustic mirror 200 is adjustable in one or more directions and one or more planes. For example, acoustic mirror 200 is rotatable about the longitudinal axis of ultrasound catheter 102 e.g., to form a ring-like treatment zone. The angle between ultrasound emitting surface 104 and a surface of acoustic mirror 200 is adjustable from an angle alpha of zero (α=0), e.g., a fully closed state, to an angle alpha (α) of 80 degrees e.g., a fully open state (angle alpha is shown, for example, in FIG. 3B).

In some embodiments, acoustic mirror 200 is rotatable in relation to ultrasound catheter 102 about a longitudinal axis of ultrasound catheter 102, to face in any desired direction radially in relation to US catheter 102.

Reference is now made to FIG. 3A, 3B and 3C, which is a perspective view (FIG. 3A) and cross-section view schematic illustrations (FIGS. 3B and 3C) of ultrasound catheter n acoustic mirror system 20 in accordance with some embodiments of the invention. In some embodiments, an acoustic mirror 200 is a cantilever-shaped mirror coupled at one end to ultrasound transducer 100 of ultrasound catheter 102. In some embodiments, acoustic mirror 200 comprises a base 302 at one end and a free edge 304 at a second end. In some embodiments, base 302 of acoustic mirror 200 is coupled to ultrasound catheter 102 proximally to ultrasound transducer 100 emitting surface 104 such that to interfere at least in part with a path of propagation of ultrasound beam 150 emitted from emitting surface 104 and deflect the beam at an angle in relation to a beam deflecting surface 306 of acoustic mirror 200. In some embodiments, and as explained elsewhere herein, acoustic mirror 200 is pivotable about base 302. In some embodiments, base 302 is resilient, e.g., a spring, a hinge or forms a hinge with a wall of ultrasound catheter 102 on which acoustic mirror 200 is mounted.

In some embodiments, acoustic mirror 200 extends over the face of transducer emitting surface 104 at an inclination angle (α), being the angle between a longitudinal axis (M) of acoustic mirror 200 and longitudinal axis (C) of US catheter 102, such that all, or partial, of the energy emitted by the transducer is deflected by the beam deflecting surface 306. In some embodiments, the geometry of beam deflecting surface 306 corresponds to the geometry of ultrasound transducer 100 emitting surface 104 so that to capture most, and possibly all of the acoustic energy emitted from ultrasound transducer 100.

As explained elsewhere herein, in some embodiments, base 302 serves as a hinge about which acoustic mirror 200 is moveable from an open state shown in FIG. 3B to a closed state as shown in FIG. 3C. For example, in some embodiments, ultrasound catheter 102 is inserted into an atrium of a subject, in a vicinity of the pulmonary vein, with acoustic mirror 200 in a closed state. Once placed in propinquity to the target area (e.g., the pulmonary vein ostia), acoustic mirror 200 is moved from the closed state to open state. In some embodiments, base 302 is resilient. In some embodiments, base 302 is biased towards the open state. In some embodiments, base 302 comprises a hinge.

In some embodiments, ultrasound transducer catheter 102 comprises an acoustic mirror control mechanism. In some embodiments, control mechanism is configured to reversibly move acoustic mirror 200 from a closed position to an open position. In some embodiments, acoustic mirror 200 control mechanism comprises a slidable retainer 402 shaped, e.g., as a slidable sleeve or ring.

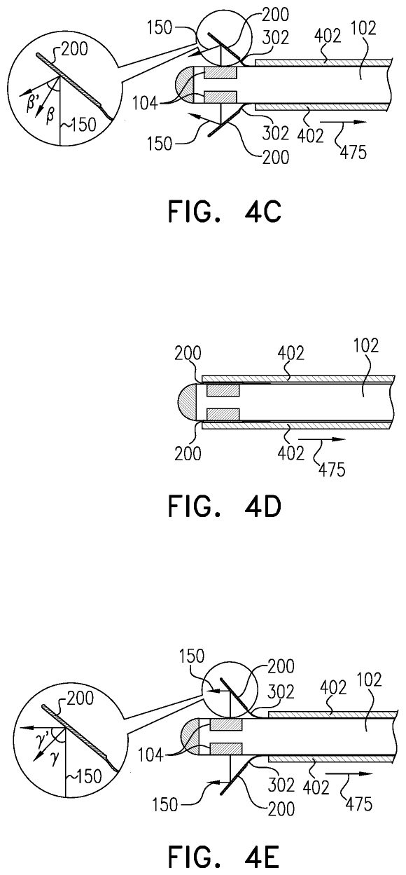

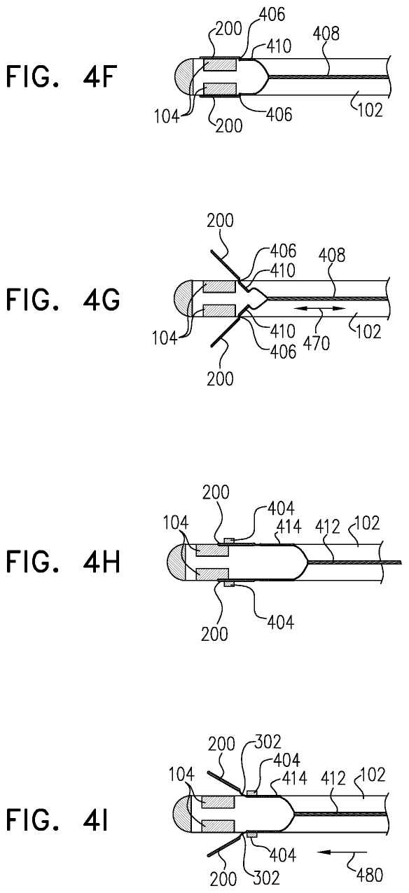

Referring now to FIGS. 4A, 4B, 4C, 4D, 4E, 4F, 4G, 4H and 4I, which are cross section schematic illustrations of two examples of an ultrasound catheter acoustic mirror system 20 comprising acoustic mirror control mechanisms, in accordance with some embodiments of the invention. In the exemplary embodiment depicted in FIGS. 4A and 4B, acoustic mirror control mechanism comprises a slidable retainer 402 shaped as a slidable sleeve. In some embodiments, acoustic mirror 200 is biased radially outwards by a resilient base 302 coupling acoustic mirror 200 to ultrasound catheter 102. In some embodiments, resilient base 302 comprises a shape memory alloy, preset to open acoustic mirror 200 to a predetermined desired state. In some embodiments, slidable retainer 402 is slidable proximally and/or distally to partially or fully expose resilient base 302 and control the angle of deflection (radial opening) of acoustic mirror 200.

In a closed state (FIG. 4A), slidable retainer 402 is positioned over acoustic mirror 200 urging acoustic mirror 200 against an external surface of transducer 100. In this orientation, resilient base 302 is placed in a spring-loaded state. Sliding slidable retainer 402, e.g., proximally uncovers acoustic mirror 200, which, urged by base 302, springs radially outward, shown in FIG. 4B and move to an open state. In the open state, acoustic mirror 200 is positioned to guide ultrasound beam 150 toward the target tissue, as described herein. In some embodiments, the moving acoustic mirror 200 between the closed and open states is reversable as indicated by double headed arrow 450, in FIG. 4A.

Referring now to FIGS. 4C-4E. FIG. 4D shows acoustic mirror 200 in a closed state thereof, with slidable retainer 402 fully positioned over the acoustic mirror. FIGS. 4C and 4E show slidable retainer 402 partially (FIG. 4C) or fully (FIG. 4E) withdrawn proximally in the direction indicated by arrow 475 to expose acoustic mirror 200. Typically, the angle of inclination of acoustic mirror 200 in relation to a surface 104, and subsequently a deflection angle of beam 150 by acoustic mirror 200, is controlled by the degree of exposure of base 302. In other words, a length of resilient base 302 is extended so as to allow gradual movement of acoustic mirror 200 from a closed state to an open state as well as control of the angle of inclination of the emitted beam 150. In some embodiments, the angle of inclination of acoustic mirror 200 in relation to a surface 104 of ultrasound transducer 100 of ultrasound catheter 102 is between 0 (zero) and 75 degrees.

For example, and as shown in FIG. 4C, slidable retainer 402 is only partially positioned over acoustic mirror 200 to partially exposes resilient base 302 allowing only partial inclination of acoustic mirror 200. A partial inclination of acoustic mirror 200 results in a sharp deflection angle beta (β), e.g., a 45-degree deflection. Further proximal movement of slidable retainer 402, as shown in FIG. 4E, fully exposes resilient base 302 allowing a greater inclination of acoustic mirror 200 resulting in a larger deflection angle gamma (γ′), being larger than deflection angle beta (β) e.g., a 90-degree deflection. Thus, in some embodiments, the control of the angle of inclination alpha (α) (shown in FIG. 3B) controls the angle of deflection e.g., angle beta (β′) and/or angle gamma (γ′) of ultrasound beam 150 emitted from the surface 104 of ultrasound transducer 100. In some embodiments, angle of inclination alpha (α) is in a range between 20 degrees to 60 degrees. However, in some embodiments, angle of inclination alpha (α) is below 20 degrees or above 60 degrees.

Referring now to FIGS. 4F and 4G, which show an additional control mechanism for acoustic mirror 200. The exemplary embodiment depicted in FIGS. 4F and 4G illustrates an acoustic mirror 200 control mechanism comprising a stepped-type hinge and a control wire coupled, e.g., to a control handle of ultrasound catheter 102. In this configuration, movement of a control wire 408 distally or proximally, is indicated by double headed arrow 470. Control wire 408 is coupled to a pivoting arm 410 on a proximal side of a stepped-hinge 406 coupling pivoting arm 410 to acoustic mirror 200. Movement of control wire 408 distally, brings about opening, and an increase in angle of inclination alpha (α) of acoustic mirror 200. Movement of control wire 408 proximally, brings about closing, and a decrease in inclination angle (α) of acoustic mirror 200.

Referring now to FIGS. 4H and 4I, which show yet another example for a control mechanism for acoustic mirror 200. As shown in the exemplary embodiment depicted in FIGS. 4H and 4I, acoustic mirror 200 together with base 302 are slidable proximally and distally within a channel 404 positioned on a periphery of a wall of ultrasound catheter 102. A control wire 412 having at least one slidable driving arm 414 is coupled at one end e.g., to a control handle of ultrasound catheter 102, and at a second end the driving arm 414 of control wire 412 is coupled to base 302 of acoustic mirror 200. In the closed state of acoustic mirror 200, control wire 412 is at a maximal proximal position trapping acoustic mirror 200 within channel 404. Movement of control wire 412 distally, as indicated by arrow 480, brings about sliding of acoustic mirror 200 and base 302 distally, freeing base 302 from channel 404 and enabling the opening, and an increase in angle of inclination (α) of acoustic mirror 200. Movement of control wire 408 proximally brings about sliding of acoustic mirror 200 and base 302 proximally, at least partially trapping base 302 inside channel 404 and the closing and decreasing in inclination angle alpha (α) of acoustic mirror 200.

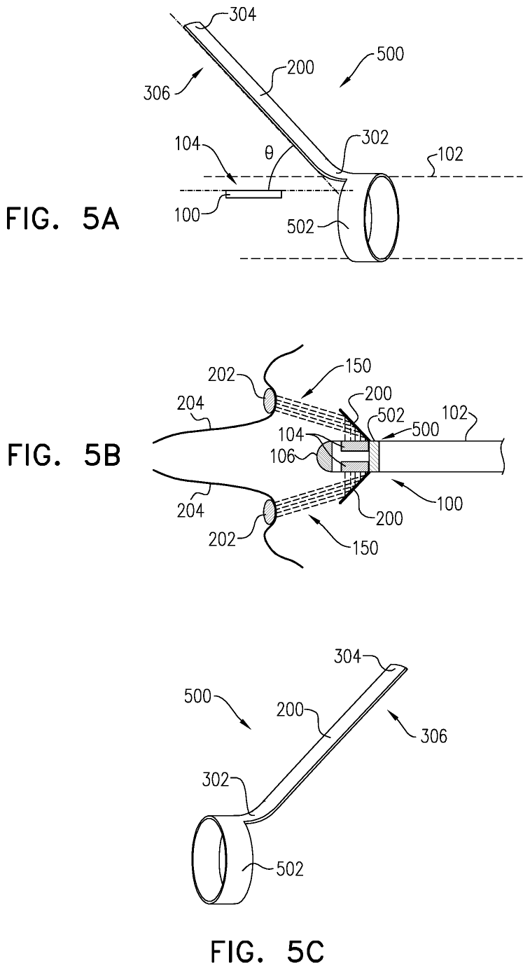

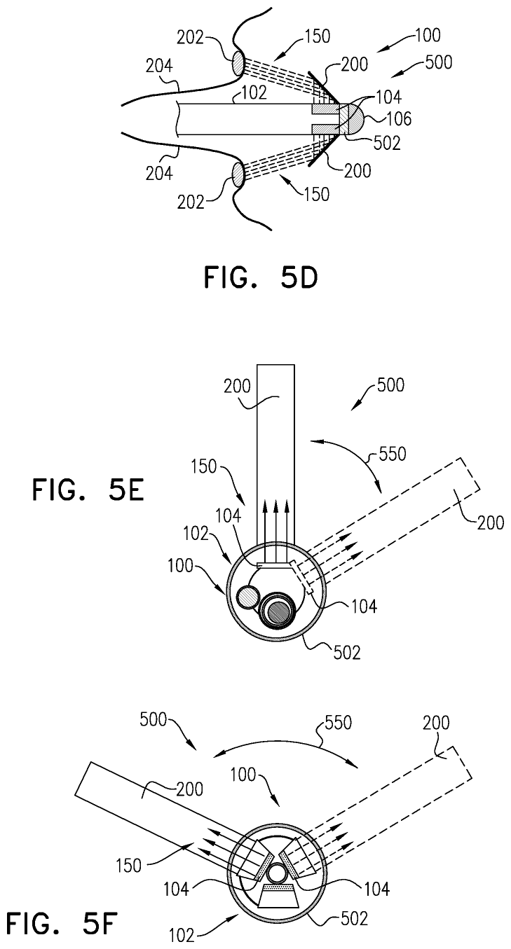

Reference is now made to FIGS. 5A, 5B, 5C, 5D, 5E and 5F, which are perspective view and cross section view schematic illustrations of an acoustic mirror add-on 500 for use with a transluminal ablation catheter such as an ultrasound catheter in accordance with some embodiments of the invention. FIGS. 5A and 5B, are perspective view schematic illustrations of an acoustic mirror add-on having a mounting ring, and FIGS. 5C and 5D, are cross section view schematic illustrations of an acoustic mirror add-on having a rotatable mounting ring, in accordance with some embodiments of the invention.

As shown in the exemplary embodiment depicted in FIG. 5A, an acoustic mirror add-on 500 comprises an acoustic mirror 200 coupled to an ultrasound catheter mounting ring 502 via a base 302. In some embodiments, acoustic mirror 200 add-on 500 is couplable to any conventional ultrasound catheter 102, drawn in FIG. 5A with phantom lines, e.g., by sliding mounting ring 502 over the catheter to a desired position. In some embodiments, other means of attachment are also employable, e.g., by a clamp, adhesive, welding, extrusion, bonding and any combination thereof.

In some embodiments, add-on 500 is fashioned as an integral add-on comprising a ring 502 and mirror 200. In some embodiments, integral add-on 500 is cut out as an integral add-on of, e.g., a tube or a similar component made of nitinol or any other suitable material. In some embodiments, ring 502 and mirror 200 are integrally formed and are made of the same material. In some embodiments, a resilience and/or springiness may be imparted to base 302 and/or any other part of add-on 500, by, e.g., any type of suitable treatment, e.g., shaping, molding, thinning, cold working, heat treating, and the like.

As explained elsewhere herein, add-on 500 is coupled to a conventional ultrasound catheter 102 in a position at which the geometry of beam deflecting surface 306 corresponds to the geometry of ultrasound transducer 100 emitting surface 104 so as to capture most, and possibly all of the acoustic energy emitted from ultrasound transducer 100.

As shown in FIG. 5A and corresponding FIG. 5B, in some embodiments, acoustic mirror add-on 500 is positioned at a proximal border of ultrasound transducer 100 such that beam deflecting surface 306 corresponds to the geometry of ultrasound transducer 100 emitting surface 104 and partially faces distally such that an ultrasound beam emitted laterally from the ultrasound emitting surface 104 of transducer 100 is deflected generally forward, passed a tip 106 of ultrasound catheter 102. In some embodiments, add-on 500 mounting ring 502 is positioned along conventional ultrasound catheter 102 such that beam deflecting surface 306 is angled at an angle (θ) in relation to ultrasound emitting surface 104 of ultrasound catheter 102 US transducer 100. In some embodiments, angle (θ) is between 0 (zero) and 75 degrees.

Acoustic mirror add-on 500 may be oriented as desired. In some embodiments, and as shown in FIG. 5C and corresponding FIG. 5D, add-on 500 is positioned at a distal border of ultrasound transducer 100 such that beam deflecting surface 306 corresponds to the geometry of ultrasound transducer 100 emitting surface 104 and partially faces proximally such that an ultrasound beam emitted laterally from the ultrasound emitting surface 104 of transducer 100 is deflected generally backward, away from tip 106 of ultrasound catheter 102.

In some embodiments, as shown in FIGS. 5E and 5F, add-on 500 is rotatable about a longitudinal axis of an ultrasound catheter 102. FIG. 5E depicts an exemplary embodiment in which add-on 500 is mounted on ultrasound catheter 102 having a rotatable ultrasound transducer 100 and ultrasound emitting surface 104. In some embodiments, rotation of ultrasound transducer 100 from a first position to a second position indicated in FIG. 5E by phantom lines, is shadowed by a corresponding reversible rotation of add-on 500, indicated by a double headed arrow 550. Rotation of add-on 500 brings acoustic mirror to a position in which the geometry of beam deflecting surface 306 corresponds to the geometry of ultrasound transducer emitting surface 104 so as to capture most, and possibly all of the acoustic energy emitted from US transducer 100.

In the exemplary embodiment depicted in FIG. 5F, add-on 500 is mounted on ultrasound catheter 102 having a plurality of ultrasound emitting surfaces 104. In some embodiments, switching ultrasound beam emission between ultrasound emitting surfaces 104 is shadowed as shown in FIG. 5F by phantom lines, by a corresponding reversible rotation of add-on 500, indicated by a double headed arrow 550. Rotation of add-on 500 brings acoustic mirror to a position in which the geometry of beam deflecting surface 306 corresponds to the geometry of ultrasound transducer emitting surface 104 so that to capture most, and possibly all of the acoustic energy emitted from ultrasound transducer 100.

A potential advantage of acoustic mirror add-on 500 and configurations thereof, is in that acoustic mirror add-on 500 provides both directional control of an ultrasound ablative beam to a conventional ultrasound catheter as well as directional versatility and flexibility in selection of approaches to an area to be treated.

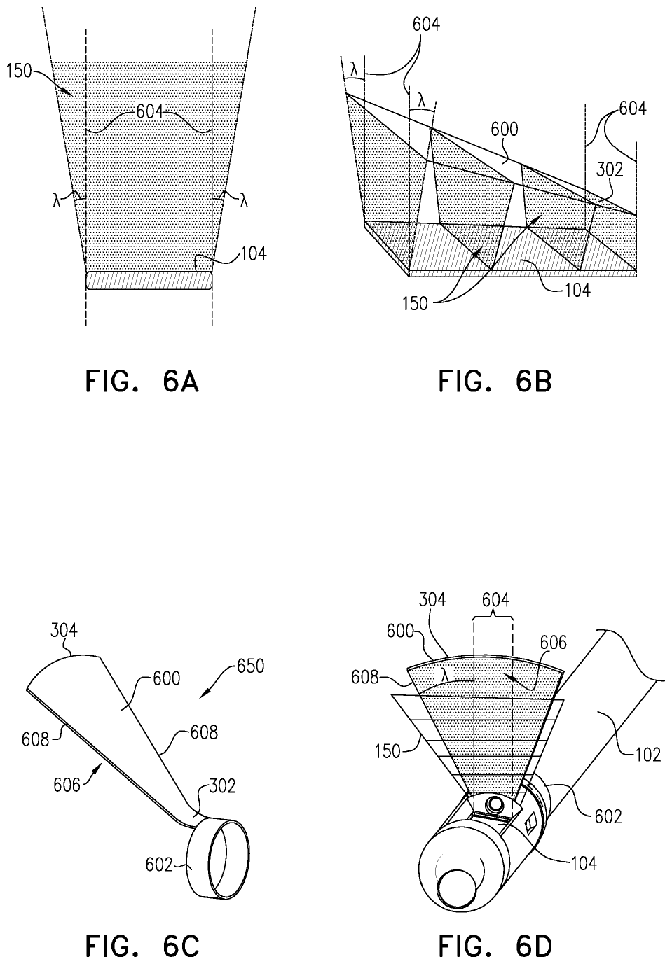

Reference is now made to FIGS. 6A, and 6B, which are cross section view and perspective view schematic illustrations of behavior of an emitted ultrasound beam and an acoustic mirror, in accordance with some embodiments of the invention. As depicted in FIGS. 6A and 6B, a propagation profile of ultrasound beam 150 emitted from emitting surface 104 of ultrasound transducer 100, planar or spherical, diverges away from borders 604 of ultrasound emitting surface 104 at a divergence angle lambda (λ). Therefore, for some applications, to ensure the capture of the entire acoustic beam 150 profile, acoustic mirror 600 has a trapezoidal flat shape that is narrow at its base 302 end and diverges outwards in relation to its longitudinal axis, increasing in width along its length, to account for the wider cross-sectional area of the acoustic beam 150 at portions distanced further away from the transducer emitting surface 104.

Reference is now made to FIGS. 6C and 6D, which are schematic illustrations of an acoustic mirror add-on 650 in accordance with additional embodiments of the invention. In some embodiments, an acoustic mirror add-on 650 comprises an acoustic mirror 600 coupled to an ultrasound catheter mounting ring 602 via a base 302. In some embodiments, acoustic mirror add-on 650 is couplable to any conventional ultrasound catheter e.g., by sliding mounting ring 602 over the catheter to a position. In some embodiments, other means of attachment are also employable, e.g., by a clamp, adhesive, welding, extrusion, bonding and any combination thereof.

As explained elsewhere herein, and shown in FIGS. 6C and 6D, acoustic mirror add-on 650 is coupled to a conventional ultrasound catheter in a position at which the geometry of beam deflecting surface 606 captures most, and possibly all of the acoustic energy emitted from ultrasound transducer 100. In some embodiments, acoustic mirror 600 is shaped as a semi-cone or parabola-like geometry being narrow at base 302 and wider at a free edge 304. In some embodiments, in view of divergence angle lambda (λ), the more distal (away from base 302 and towards edge 304) the impingement of ultrasound beam 150 on deflecting surface 606 of acoustic mirror 600 the greater the distance between acoustic mirror 600 and ultrasound emitting surface 104 and the area (e.g., width) of impingement increases. Hence, in some embodiments, side-edges 608 of acoustic mirror 600 diverge laterally outwards at a divergence angle corresponding to divergence angle lambda (λ) of US beam 150 from borders 604 of ultrasound emitting surface 104.

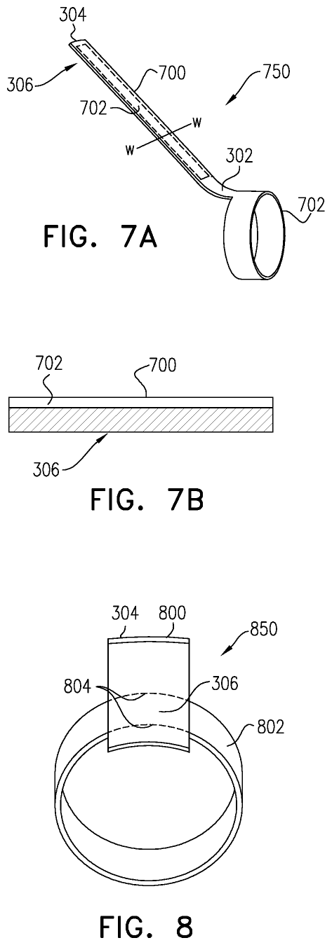

Reference is now made to FIGS. 7A and 7B, which are a perspective view and cross-section view of an acoustic mirror add-on 750, in accordance with some embodiments of the invention. For some embodiments, any of the acoustic mirrors described herein (either the acoustic mirrors that are integrated with an ultrasound catheter as shown in FIG. 3B, or an acoustic mirror that is a component of an acoustic mirror add-on such as add-ons 500 and 650), may additionally comprise an air chamber, such as air chamber 702 shown in FIG. 7A-B. FIGS. 7A show acoustic mirror add-on 750 comprising acoustic mirror 700 and 7B, shows a cross section through acoustic mirror 700 along a W-W axis. Acoustic mirror 700 can be shaped as any one of the acoustic mirrors (e.g., 200, 600) described elsewhere herein and comprises an air chamber 702. Alternatively, and optionally, acoustic mirror 200, 600 is encapsulated with air to increase energy reflection or deflection and prevent energy absorption within the mirror material.

In some embodiments, air chamber 702 comprises a buffering zone that provides a high impedance mismatch with blood and blocks ultrasonic energy from crossing through acoustic mirror 700 and directs the ultrasound energy impinging on surface 306 away from surface 306.

In some embodiments, to effectively deflect the transmitted acoustic beam 150 acoustic mirror 700 comprises a material of high impedance difference compared to blood. Alternatively, and optionally, acoustic mirror 700 comprises a plurality of layers with significant impedance mismatch therebetween to reduce acoustic refraction or absorbent within acoustic mirror 700. Such as, for example, air chamber 702 or acoustic mirror 700 comprises stainless steel having a high impedance mismatch with blood.

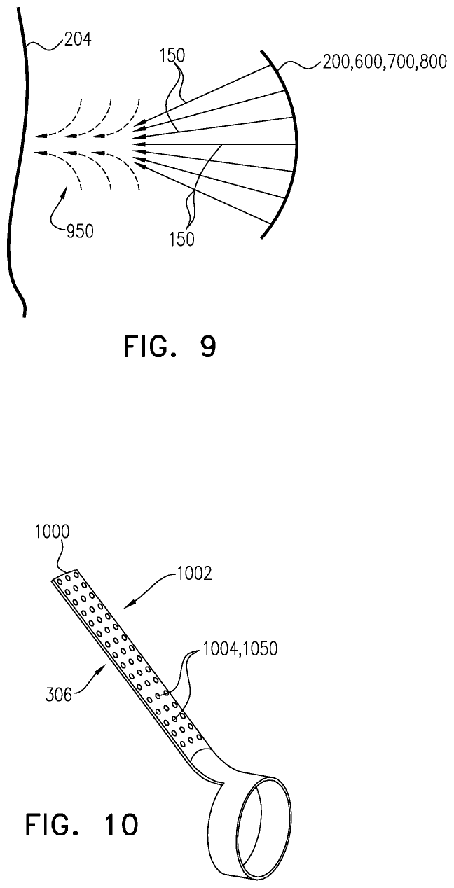

In some embodiments, and as explained elsewhere herein, an acoustic mirror add-on 850 comprises an acoustic mirror 800, similar to acoustic mirrors 200, 600 and 700 described elsewhere herein, and coupled to an ultrasound catheter mounting ring 802. In some embodiments, acoustic mirror 800 is concave along its short axis 804, e.g., as shown in FIG. 8, so that to focus ultrasound beam 150 reflected off deflecting surface 306 (as shown in FIG. 9). The focusing of the energy intensifies the deflected beam 150 and overcomes energy loss during the deflection of the transmitted beam 150 off deflecting surface 306.

Reference is now made to FIG. 9, which is a cross-section view schematic illustration of a concave deflective surface induced jet effect in accordance with some embodiments of the invention. In some embodiments, a concave deflective surface 306 acts as a collimator, collimating ultrasound beam 150 as it is reflected or deflected off deflecting surface 306 in a direction of tissue 204 to be treated.

In some embodiments, collimated beam energy reflected off a concave surface generates a jet effect 950 in surrounding blood stream maintaining the same temperature as that of the surrounding blood stream. In some embodiments, the collimated beam energy is above 50 W/cm∧2. In some embodiments, the collimated beam energy is above 70 W/cm∧2. In some embodiments, the collimated beam energy is above 90 W/cm∧2.

A potential advantage in such a jet effect 950 is in that a jet aimed at a treatment area cools the tissue 204 wall at the point of penetration of the ultrasound beam into the tissue and prevents tissue charring. However, in some embodiments, a jet effect 950 may cause acoustic mirror 200, 600, 700, 800 to vibrate and heat up to a temperature above circumambient blood temperature eliciting a blood coagulation sequence. For some such applications, the apparatus and methods described with reference to FIG. 10 are used.

Reference is now made to FIG. 10, which is a schematic illustration of an acoustic mirror add-on 1000 for use with a transluminal ablation catheter, the acoustic mirror add-on comprising a cooling mechanism, in accordance with some embodiments of the invention.

In some embodiments, acoustic mirror add-on 1000, depicted for example in FIG. 10, which is a perspective view schematic illustration of an acoustic mirror cooling mechanism, in accordance with some embodiments of the invention, comprises one or more cooling mechanisms to prevent or reduce heating of the acoustic mirror surfaces and maintain a temperature equal to or below circumambient blood temperature. In some embodiments, a back surface 1002 or the edges of an acoustic mirror add-on 1000 comprise a heat sink 1050. In some embodiments, heat sink 1050 comprises protrusions (e.g., fins), voids 1004 or a combination thereof that increase the surface area of acoustic mirror add-on 1000 and increase effective heat dissipation than surfaces devoid of heat sinks.

Alternatively, and optionally, acoustic mirror add-on 1000 comprises cooling fluid channels that continuously circulate cooling fluid along and deep to the surface. In some embodiments, one or more surfaces and/or edges of acoustic mirror add-on 1000 are coated with materials having a high heat conductivity coefficient e.g., gold. In some embodiments, one or more surfaces of acoustic mirror add-on 1000 are coated with anticoagulant materials.

In some embodiments, acoustic mirror add-on 1000 is encapsulated having an inlet in fluid communication with a source of cooling fluid and an outlet, configured to circulate cooling fluid around acoustic mirror add-on 1000 dissipating heat from one or more surfaces and/or edges of acoustic mirror add-on 1000.

Although the system and techniques of embodiments the present invention have generally been described herein as being applied to cardiac tissue, these techniques may additionally be used, mutatis mutandis, to treat any other lumen, cavity or tissue of a subject, e.g., a uterus. For some embodiments, these system and techniques may additionally be used, mutatis mutandis, to treat any blood vessel including but not limited to a renal artery, carotid artery, jugular vein, or splenic vein and/or or aorta of a subject. For some embodiments, the system and techniques may additionally be used, mutatis mutandis, to treat the lungs and lung vasculature, and/or nasal airways.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An acoustic mirror for use with an ultrasound catheter that includes an ultrasound beam emitting surface, comprising:
an ultrasound beam deflecting surface; and
a base couplable to the ultrasound catheter,
wherein when the acoustic mirror is mounted on the ultrasound catheter, the deflecting surface is angled in relation to the ultrasound beam emitting surface and configured to:
(i) at least partially interfere with a path of propagation of an ultrasound beam emitted from the emitting surface; and
(ii) deflect the ultrasound beam impinging on the deflecting surface.

2. The acoustic mirror according to claim 1, wherein the ultrasound beam deflecting surface is pivotable about the base.

3. The acoustic mirror according to claim 1 wherein the base is resilient.

4. The acoustic mirror according to claim 1, wherein a geometry of the ultrasound beam deflecting surface at least partially overlaps a geometry of the ultrasound beam emitting surface.

5. The acoustic mirror according to claim 1, wherein when mounted on the ultrasound catheter, the deflecting surface is pivotable about the base from a closed state in which the deflecting surface is parallel to a wall of the ultrasound catheter, to an open state in which the deflecting surface is angled in relation to the emitting surface.

6. The acoustic mirror according claim 1, wherein the deflecting surface is pivotable about the base such that an angle between the deflecting surface and the emitting surface is changeable at least from 0 (zero) to 75 degrees.

7. The acoustic mirror according to claim 1, wherein the deflecting surface defines a short axis and is concave along its short axis.

8. The acoustic mirror according to claim 1, wherein the acoustic mirror further comprises an air chamber.

9. The acoustic mirror according to claim 1, wherein the acoustic mirror further comprises a cooling mechanism.

10. The acoustic mirror according to claim 9, wherein the cooling mechanism comprises at least one or a combination of a heat sink, a cooling fluid channel, a coating of a material having a high heat conductivity coefficient and a cooling capsule.

11. The acoustic mirror according to claim 1, wherein the acoustic mirror is coated with an anticoagulant.

12. The acoustic mirror according to claim 1, wherein the acoustic mirror is encapsulated in air to increase energy reflection and inhibit energy absorbent within the mirror material.

13. The acoustic mirror according to claim 1, wherein the deflecting surface has a semi-cone or parabola-like geometry.

14. The acoustic mirror according to claim 1, wherein side-edges of the acoustic mirror diverge laterally outwards at a divergence angle corresponding to a divergence angle of an ultrasound beam from borders of the ultrasound emitting surface.

15. An acoustic mirror add-on for use with ultrasound catheter that includes a source of ultrasound energy and an ultrasound beam emitting surface, comprising:
an ultrasound beam deflecting surface;
an ultrasound catheter mounting ring couplable to the ultrasound catheter; and
a base coupling the deflecting surface and the mounting ring; and
wherein the deflecting surface is angled in relation to the emitting surface so that when the acoustic mirror is positioned such that the deflecting surface faces the source of ultrasound energy and at least partially interferes with a path of propagation of an ultrasound beam emitted by the emitting surface, the ultrasound beam is deflected from the path of propagation.

16. The acoustic mirror add-on according to claim 15, wherein the base is resilient.

17. The acoustic mirror add-on according to claim 15, wherein the mounting ring is mountable on a standard ultrasound catheter.

18. The acoustic mirror add-on according to claim 15, wherein the mounting ring is slidingly mountable on a standard ultrasound catheter.

19. The acoustic mirror add-on according to claim 15, wherein the mounting ring is rotatable about a longitudinal axis of the ultrasound catheter.

20. The acoustic mirror add-on according to claim 15, wherein the mounting ring is mountable on an ultrasound catheter having a rotating ultrasound transceiver and is configured to rotate about the ultrasound catheter in correspondence with rotation of the ultrasound transceiver.

21. The acoustic mirror add-on according to claim 15, wherein the mounting ring is slidable along the ultrasound catheter.

22. The acoustic mirror add-on according to claim 21, wherein an angle between the deflecting surface and the emitting surface is between 0 and 75 degrees.

23-40. (canceled)

41. Apparatus for use with a lumen of a subject that extends from a chamber of a heart of the subject, the apparatus comprising:
a transluminal ablation catheter comprising at least one ultrasound transducer (i) having at least one ultrasound beam emitting surface, and (ii) configured to be inserted into the chamber of the subject's heart and to emit an ultrasound beam from the ultrasound beam emitting surface; and
an ultrasound beam guiding element coupled to the transluminal ablation catheter and comprising an ultrasound beam guiding surface configured to be positioned in relation to the ultrasound beam emitting surface so as to at least partially interfere with a path of propagation of the emitted ultrasound beam such that the ultrasound beam is directed from the guiding element toward an ostium of the lumen to ablate tissue of the ostium of the lumen.

42. The apparatus according to claim 41, wherein the ultrasound beam emitting surface is configured such that ultrasound beam is directed from the ultrasound beam guiding surface from a distance that is within 3-6 mm from the tissue of the ostium of the lumen.

43. The apparatus according to claim 41, wherein the at least one ultrasound transducer is configured to be inserted into a left atrium in a vicinity of a pulmonary vein ostium and is configured to ablate tissue of an ostium of the pulmonary vein, to thereby electrically isolate the pulmonary vein.
